(19) United States
(12) Patent Application Publication
(10) Pub. No.: US 2011/0288235 A1
(43) Pub. Date: Nov. 24, 2011

(54) Process for the Preparation of Pramlintide

(75) Inventors: Tsung Yu Hsiao, Kaohsiung County (TW); Jin Guo Ding, Minhang (CN)

(73) Assignee: ScinoPharm Taiwan Ltd., Tainan County (TW)

(21) Appl. No.: 13/113,354

(22) Filed: May 23, 2011

Related U.S. Application Data
(63) Continuation-in-part of application No. 12/553,567, filed on Sep. 3, 2009, now abandoned.
(60) Provisional application No. 61/190,928, filed on Sep. 3, 2008.

(51) Int. Cl. C07K 17/08; C07K 7/06; C07K 14/575

(52) U.S. Cl. 525/54.11; 530/324; 530/328; 530/327

(57) ABSTRACT

The present invention provides for an efficient process for making pramlinitide, as well as novel intermediates for the making of the same.

### RELATED APPLICATIONS

This application is a continuation-in-part application of U.S. Ser. No. 12/553,567, filed Sep. 3, 2009.

### BACKGROUND OF THE INVENTION

1. Field of the Invention

The present invention relates to the efficient commercial synthesis for the making of pramilintide, a synthetic analog of human amylin which is a peptide hormone. Pramilintide is indicated to treat type 1 and type 2 diabetics who use insulin. The process for making pramilintide substantially comprises the syntheses of various fragments of the polypeptide and the coupling of the fragments to produce pramilintide.

2. Description of the Related Arts

The preparation and use of pramlinitide is disclosed in U.S. Pat. No. 5,686,411, which is herein incorporated in its entirety by reference. Pramlintide is known to be prepared by solid phase synthesis that successively adds the desired amino acid to a growing peptide chain. Typically, an α-N-carbamoyl protected amino acid and an amino acid attached to the growing peptide chain on a resin support are reacted at room temperature in an inert solvent in the presence of coupling agents such as dicyclohexylcarbodiimide 1-hydroxybenzotriazole in the presence of a base. The α-N-carbamoyl protecting group is removed from the resultant peptide with a reagent such as trifluoroacetic acid or piperidine, and the coupling reaction repeated with the next desired N-protected amino acid. Suitable N-protecting groups are known in the art, with t-butyloxycarbonyl herein preferred. U.S. Pat. No. 5,424,394 provides a classical stepwise approach for the synthesis of amylin and amylin analogues. Single amino acid residues are covalently coupled to a growing peptide chain which is covalently linked to a solid resin support. The synthetic route is very lengthy and inefficiently since several coupling and deprotected steps have to be repeated. The present invention provides a more efficient synthesis of pramlintide and the yield and purity of final product will be improved in view of the prior art.

### SUMMARY OF THE INVENTION

The present invention provides for an efficient process for making pramlinitide that is high in yield and scalable for commercial production. The process comprises the stepwise synthesis of amino acid segments, and the coupling together of these segments to produce pramlinitide. The present invention provides for four novel intermediate amino acid segments for the preparation of pramlintide. In a preferable embodiment of the invention, the four segments are synthesized in solid phase synthesis and the coupling reaction is performed in solid phase, too. Preferably, the segments are produced by coupling a protected designated amino acid to a growing peptide chain that is covalently linked to an insoluble solid resin support. The “protected designated amino acid” refers to single amino acid (having protected sidechains and amino termini) which generally proceeds from the carboxy-terminal end to the amino-terminal end to give a peptide of specified sequence. A “growing peptide chain” refers to a general cycle of synthesis comprising deprotection of the α-amino group of the resin-bound amino acid or peptide, followed by reaction (coupling) of the free α-amino group with some carboxyl-activated form of the next α-amino protected amino acid to form a peptide linkage and to give a support-bound peptide.

### DETAILED DESCRIPTION OF THE INVENTION

To assist in understanding the present invention, the following examples are included which describe the results of a series of experiments. The following examples relating to this invention should not, of course, be construed as specifically limiting the invention. Such variations of the invention, now known or later developed, which would be within the purview of one skilled in the art are considered to fall within the scope of the present invention as hereinafter claimed.

### Example 1

### Preparation of Protected Fragment S1 (1-8)

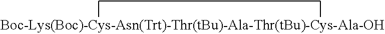

Synthesis of the protected peptide was carried out by a stepwise Fmoc SPPS (solid phase peptide synthesis) procedure starting with loading a Fmoc-Ala-OH to 2-Cl-Trt-Cl resin (CTC resin), The CTC resin (4 g) after washing was stirred with a solution of Fmoc-Ala-OH (1.49 g) in DMF in the presence of diisopropylethylamine (DIEA, 2.3 g) for 1.5 h. The resin was further capped by treatment with mixed solvent of 5:4:1 volume ratio of DCM/MeOH/DIEA for 0.5 h. After washing of the resin the Fmoc protecting group was removed by treatment with 20% piperidine in DMF twice for 10 min and 30 min, respectively. After washing of residual reagents the second amino acid (Fmoc-Cys (Acm)-OH) was introduced to start the first coupling step. The Fmoc protected amino acid was activated in situ using 1:1:2 molar ratio of HBTU (O-Benzotriazole-N,N,N′,N′-tetramethyl-uronium-hexafluoro-phosphate)/HOBt (N-Hydroxybenzotriazole)/DIEA in DMF and subsequently coupled to the growing peptide on resin for 3 h. Completion of the coupling was indicated by a Kaiser test. After washing of the resin, the Fmoc protecting group on the α-amine was removed with 20% piperidine in DMF twice for 10 min and 30 min, respectively. These steps were repeated each time with another amino acid according to peptide sequence. All amino acids used were Fmoc-N^{α} protected except the last amino acid in the sequence, Boc-Lys(Boc)-OH. Trifunctional amino acids were side chain protected as follows: Lys(Boc)-OH, Asn(Trt)-OH, Thr(tBu)-OH. Three equivalents of the activated amino acids were employed in the coupling reactions. At the end of the synthesis the peptide resin was washed with DMF, MeOH followed by MTBE, and dried under vacuum to give dry peptide on resin.

The peptide was cleaved from the peptide on resin (8 g) prepared as described above, using 20% TFE solution in DCM for 2 h. The peptide solution was solvent replaced by MeOH and concentrated (30 mL). The concentrated residue was cooled and the product was precipitated by adding water (30 mL). The precipitated product was separated by filtration and washed with mixed solvent of MeOH/water (10 mL/10 mL) twice to give Boc-Lys(Boc)-Cys(Acm)-Asn(Trt)-Thr(tBu)-Ala-Thr(tBu)-Cys(Acm)-Ala-OH(S1a).

10 g S1a was dissolved in a mixed solvent of DMF/pyridine/MeOH, and then add a solution with I₂ in DMF/MeOH during 2.5 h. After 1 h, the mixture was quenched with Vitamin C/ammonium acetate in water. More water was added to gain crude S1. Further purification was carried out by silica gel to give S1

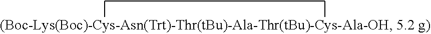

### Example 2

### Preparation of Protected Fragment S2 (9-19)

### Fmoc-Thr(tBu)-Gln(Trt)-Arg(Pbf)-Leu-Ala-Asn(Trt)-Phe-Leu-Val-His(Trt)-Ser(tBu)-OH

Synthesis of the protected peptide was carried out by a stepwise Fmoc SPPS (solid phase peptide synthesis) procedure starting with loading a Fmoc-Ser(tBu)-OH to 2-Cl-Trt-Cl resin (CTC resin). The CTC resin (10 g) after washing was stirred with a solution of Fmoc-Ser(tBu)-OH (4.6 g) in DMF in the presence of diisopropylethylamine (2.3 g) for 1.5 h. After washing of the resin the Fmoc protecting group was removed by treatment with 20% piperidine in DMF twice for 10 min and 30 min, respectively. After washing of residual reagents the second amino acid (Fmoc-His(Trt)-OH) was introduced to start the first coupling step. The Fmoc protected amino acid was activated in situ using 1:1:2 molar ratio of HBTU/HOBt/DIEA in DMF and subsequently coupled to the growing peptide on resin for 3 h. Completion of the coupling was indicated by a Kaiser test. After washing of the resin, the Fmoc protecting group on the α-amine was removed with 20% piperidine in DMF twice for 10 min and 30 min, respectively. These steps were repeated each time with another amino acid according to peptide sequence. All amino acids used were Fmoc-N^{α} protected. Trifunctional amino acids were side chain protected as follows: Asn(Trt)-OH, Arg(Pbf)-OH, Gln(Trt)-OH and Thr(tBu)-OH. Three equivalents of the activated amino acids were employed in the coupling reactions. At the end of the synthesis the growing peptide on resin was washed with DMF, MeOH followed by MTBE, and dried under vacuum to give dry peptide on resin.

The peptide was cleaved from the peptide on resin (24 g) prepared as described above, using 20% TFE solution in DCM for 2 h. The peptide solution was solvent replaced by MeOH and concentrated (50 mL). The product was absolutely precipitated by adding cool MeOH (50 mL) to the concentrated residue. The product was separated by filtration and washed with cool MeOH (20 mL) twice to give S2 (Fmoc-Thr(tBu)-Gln(Trt)-Arg(Pbf)-Leu-Ala-Asn(Trt)-Phe-Leu-Val-His(Trt)-Ser(tBu)-OH, 12 g).

### Example 3

### Preparation of Protected Fragment S3 (20-29)

### Fmoc-Ser(tBu)-Asn(Trt)-Asn(Trt)-Phe-Gly-Pro-Ile-Leu-Pro-Pro-OH

Synthesis of the protected peptide was carried out by a stepwise Fmoc SPPS (solid phase peptide synthesis) procedure starting with loading a Fmoc-Pro-OH to 2-Cl-Trt-Cl resin (CTC resin). The CTC resin (3 g) after washing was stirred with a solution of Fmoc-Pro-OH (1.2 g) in DMF in the presence of diisopropylethylamine (2.3 g) for 1.5 h. After washing of the resin the Fmoc protecting group was removed by treatment with 20% piperidine in DMF twice for 10 min and 30 min, respectively. After washing of residual reagents the second amino acid (Fmoc-Pro-OH) was introduced to start the first coupling step. The Fmoc protected amino acid was activated in situ using 1:1:2 molar ratio of HBTU/HOBt/DIEA in DMF and subsequently coupled to the growing peptide on resin for 3 h. Completion of the coupling was indicated by a Kaiser test. After washing of the resin, the Fmoc protecting group on the α-amine was removed with 20% piperidine in DMF twice for 10 min and 30 min, respectively. These steps were repeated each time with another amino acid according to peptide sequence. All amino acids used were Fmoc-N^{α} protected. Trifunctional amino acids were side chain protected as follows: Ser(tBu)-OH, and Asn(Trt)-OH. Three equivalents of the activated amino acids were employed in the coupling reactions. At the end of the synthesis the growing peptide on resin was washed with DMF, MeOH followed by MTBE, and dried under vacuum to give dry peptide on resin.

The peptide was cleaved from the growing peptide on resin (10 g) prepared as described above, using 1% TFA solution in DCM for 1.5 h. After neutralizing with Pyridine the peptide solution was concentrated (15 mL). The product was precipitated by adding the concentrated residue into Heptanes (50 mL). The product was separated by filtration and washed with mixed solvent of DCM/Heptanes (1 mL/3 mL) three times to give S3 (Fmoc-Ser(tBu)-Asn(Trt)-Asn(Trt)-Phe-Gly-Pro-Ile-Leu-Pro-Pro-OH, 5.2 g).

### Example 4

### Preparation of Protected Fragment S4 (30-36)

### Fmoc-Thr(tBu)-Asn(Trt)-Val-Gly-Ser(tBu)-Asn(Trt)-Thr(tBu)-OH

Synthesis of the protected peptide was carried out by a stepwise Fmoc SPPS (solid phase peptide synthesis) procedure starting with loading a Fmoc-Thr(tBu)-OH to 2-Cl-Trt-Cl resin (CTC resin). The CTC resin (10 g) after washing was stirred with a solution of Fmoc-Thr(tBu)-OH (4.8 g) in DMF in the presence of diisopropylethylamine (2.3 g) for 1.5 h. After washing of the resin the Fmoc protecting group was removed by treatment with 20% piperidine in DMF twice for 10 min and 30 min, respectively. After washing of residual reagents the second amino acid (Fmoc-Asn(Trt)-OH) was introduced to start the first coupling step. The Fmoc protected amino acid was activated in situ using 1:1:2 molar ratio of HBTU/HOBt/DIEA in DMF and subsequently coupled to the growing peptide on resin for 3 h. Completion of the coupling was indicated by a Kaiser test. After washing of the resin, the Fmoc protecting group on the α-amine was removed with 20% piperidine in DMF twice for 10 min and 30 min, respectively. These steps were repeated each time with another amino acid according to peptide sequence. All amino acids used were Fmoc-N^{α} protected. Trifunctional amino acids were side chain protected as follows: Ser(tBu)-OH, Asn(Trt)-OH and Thr(tBu)-OH. Three equivalents of the activated amino acids were employed in the coupling reactions. At the end of the synthesis the growing peptide on resin was washed with DMF, MeOH followed by MTBE, and dried under vacuum to give dry peptide on resin.

The peptide was cleaved from the peptide on resin (23 g) prepared as described above, using 20% TFE solution in DCM for 2 h. The peptide solution was solvent replaced by MeOH and concentrated (60 mL). The product was precipitated by adding MeOH (50 mL) to the concentrated residue. The product was separated by filtration and washed with MeOH/water (20 mL) twice to give S4 (Fmoc-Thr(tBu)-Asn(Trt)-Val-Gly-Ser(tBu)-Asn(Trt)-Thr(tBu)-OH, 10.6 g)

### Example 5

### Coupling and De-Fmoc Reaction

S4 (FmocThr(tBu)Asn(Trt)ValGlySer(tBu)Asn(Trt)Thr(tBu)OH) (1.0 kg; 1.0 Eq.), H-Tyr(tBu)-NH₂ (0.45 Kg; 3.0 eq.) and 1-hydroxy-7-azabenzotriazole (HOAt) (0.26 Kg; 3.0 eq) are charged into a suitable reactor under nitrogen. 1-methyl-2-pyrrolidinone (NMP) (20.7 Kg) is charged and stirred for 0.5 hr. The resulting mixture is cooled to 0 to 10° C. Then the solution of N,N-diisopropylcarbodiimide (DIC) (0.24 Kg; 3.0 eq) and 1-methyl-2-pyrrolidinone (NMP) (10.3 Kg) is dropped into the cooled resulting mixture for 1 hr while maintaining the temperature at 0 to 10° C.

The reaction mixture is warmed to 20 to 30° C. and kept for 15 hr. Diethylamine (DEA) (0.42 Kg; 10.0 eq.) is charged while maintaining the temperature at 25° C. The reaction mixture is stirred at 20 to 30° C. for 2 hr. Ethyl acetate (EA) (7.38 Kg) and softened potable water (SPW) (50.0 Kg) are slowly added to the reaction mixture while maintaining the temperature at 35° C. until the cloud point is observed and held at cloud point for 1 hr. The remained SPW is added while maintaining the temperature at 35° C. The solid is filtered and washed with mixed solvent MeOH/SPW=1/1 twice and n-heptane twice. The wet cake is purged by nitrogen for 1 hr and dried at 50° C. for 5 hr to get M2 (Thr(tBu)Asn(Trt)ValGlySer(tBu)Asn(Trt)Thr(tBu)Tyr(tBu)-NH₂) (about 0.81 Kg).

### Example 6

### Coupling Reaction

M2 (0.81 Kg; 1.0 eq), S3 (Fmoc-Ser(tBu)Asn(Trt)Asn(Trt)PheGlyProIleLeuProProOH) (0.85 Kg; 0.9 eq) and 1-hydroxy-7-azabenzotriazole (HOAt) (0.21 Kg; 3.0 eq) are charged into a suitable reactor under nitrogen. Then 1-methyl-2-pyrrolidinone (NMP) (16.7 Kg) is charged and stirred for 0.5 hr. The resulting mixture is cooled to 0 to 10° C.

Ethyl(3-dimethylaminopropyl)carbodiimide hydrochloride (EDCI) (0.30 Kg; 3.0 eq) is charged into the resulting mixture while maintaining the temperature at 20 to 30° C. and stirred for 3 hr. Ethyl acetate (EA) (5.1 Kg) and SPW (43.6 Kg) are slowly added into the reaction mixture while maintaining the temperature at 35° C. until the cloud point is observed and held at cloud point for 1 hr. The remained SPW is added while maintaining the temperature at 35° C. The solid is filtered and washed by MeOH twice. The wet cake is purged with nitrogen for 1 hr and dried at 50° C. for 5 hr to get M3 (FmocSer(tBu)Asn(Trt)Asn(Trt)PheGlyProIleLeuProProThr(tBu)Asn(Trt)ValGly Ser(tBu)Asn(Trt)Thr(tBu)Tyr(tBu)-NH₂) (about 1.56 Kg).

### Example 7

### DeFmoc Reaction

M3 (1.56 Kg; 1.0 eq) and dichloromethane (DCM) (12.5 Kg) are charged into a suitable reactor. Then piperidine (0.60 Kg; 15.0 eq) is charged while maintaining the temperature at 20 to 30° C. and stirred for 2 hr. Methyl-t-butyl ether (MTBE) (34.7 Kg) is slowly added while maintaining the temperature at 30° C. until the cloud point is observed and held at cloud point for 1 hr. The remained MTBE is added while maintaining the temperature at 30° C. The product is filtered and washed with mixed solvent of MeOH/SPW=1/1 twice and methyl-t-butyl ether (MTBE) twice. The wet cake is purged with nitrogen for 1 hr and dried at 50° C. for 5 hr to provide M4 (Ser(tBu)Asn(Trt)Asn(Trt)PheGlyProIleLeuProProThr(tBu)Asn(Trt)ValGlySer(tBu)Asn(Trt)Thr(tBu)Tyr(tBu)-NH₂) (about 1.32 Kg).

### Example 8

### Coupling Reaction

M4 (1.32 Kg; 1.0 eq), S2 (FmocThr(tBu)Gln(Trt)Arg(Pbf)LeuAlaAsn(Trt)PheLeuValHis(Trt)Ser(tBu)OH) (1.03 Kg; 0.95 eq) and 1-hydroxy-7-azabenzotriazole (HOAt) (0.17 Kg; 3.0 eq) are charged into a suitable reactor under nitrogen. Then 1-methyl-2-pyrrolidinone (NMP) (27.17 Kg) is charged and stirred for 0.5 hr. The resulting mixture is cooled to 0 to 10° C.

The solution of N,N-diisopropylcarbodiimide (DIC) (0.16 Kg; 3.0 eq) and 1-methyl-2-pyrrolidinone (NMP) (6.80 Kg) is dropped to the cooled resulting mixture for 1 hr while maintaining the temperature at 0 to 10° C. The reaction mixture is warmed to 20 to 30° C. and kept for 15 hr. Ethyl acetate (EA) (9.47 Kg) is charged into the reaction mixture. Mixed solvent of MeOH/SPW=1/1 (86.8 Kg) is slowly added while maintaining the temperature at 35° C. until the cloud point is observed and held at cloud point for 1 hr. The remained SPW is added while maintaining the temperature at 35° C. The solid is filtered and washed by mixed solvent of MeOH/SPW=411. The wet cake is purged with nitrogen for 1 hr and dried at 50° C. for 5 hr to provide M5 (FmocThr(tBu)Gln(Trt)Arg(Pbf)LeuAlaAsn(Trt)PheLeuValHis(Trt)Ser(tBu)Ser(tBu)Asn(Trt)Asn(Trt)PheGlyProIleLeuProProThr(tBu)Asn(Tr)ValGly Ser(tBu)Asn(Trt)Thr(tBu)Tyr(tBu)-NH₂) (about 2.16 Kg)

### Example 9

### De-Fmoc Reaction

M5 (2.16 Kg; 1.0 eq) and dichloromethane (DCM) (28.7 Kg) are charged into a suitable reactor under nitrogen. Then piperidine (0.32 Kg; 10.0 eq) is charged while maintaining the temperature at 20 to 30° C. and stirred for 2 hr. Methyl-t-butyl ether (MTBE) (47.9 Kg) is slowly added while maintaining the temperature at 0 to 10° C. until the cloud point is observed and held at cloud point for 1 hr. The remained MTBE is added while maintaining the temperature at 0 to 10° C. The product is filtered and washed with mixed solvent of MeOH/SPW=4/1 twice and methyl-t-butyl ether (MTBE) twice. The wet cake is purged with nitrogen for 1 hr and dried at 50° C. for 5 hr to provide M6 (Thr(tBu)Gln(Trt)Arg(Pbf)LeuAlaAsn(Trt)PheLeuValHis(Trt)Ser(tBu)Ser(tBu)Asn(Trt)Asn(Trt)PheGlyProIleLeuProProThr(tBu)Asn(Trt)ValGlySer(tBu)Asn(Trt)Thr(tBu)Tyr(tBu)-NH₂) (about 1.87 Kg).

### Example 10

### Coupling Reaction

M6 (1.87 Kg; 1.0 eq.), S1

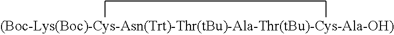

(0.46 Kg, 1.0 eq) and 1-hydroxy-7-azabenzotriazole (HOAt) (0.14 Kg; 3.0 eq.) are charged into a suitable reactor under nitrogen. 1-Methyl-2-pyrrolidinone (NMP) (19.3 Kg) is charged and stirred for 0.5 hr. The resulting mixture is cooled to 0 to 10° C.

The solution of N,N-diisopropylcarbodiimide (DIC) (0.13 Kg; 3.0 eq.) and 1-methyl-2-pyrrolidinone (NMP) (9.63 Kg) is dropped to the cooled resulting mixture for 1 hr while maintaining the temperature at 0 to 10° C. The reaction mixture is kept at 0 to 10° C. for 0.5 hr, followed by warmed to 20 to 30° C. and stirred for 17 hr. Ethyl acetate (EA) (8.39 Kg) is charged into the reaction mixture. SPW (74.6 Kg) is slowly added while maintaining the temperature at 35° C. until the cloud point is observed and held at cloud point for 1 hr. The remained SPW is added while maintaining the temperature at 35° C. for 2 hr. The solid is filtered and washed with mixed solvent of MeOH/SPW=411 three times.

The wet cake is purged with nitrogen for 1 hr and dried at 30° C. to get M7(Boc-S1S2S3S4Try(tBu)NH2) (about 2.09 Kg).

### Example 11

### Deblocking Reaction

M7 (2.09 Kg) is charged into reactor I under nitrogen. The solid is kept at temperature 0 to 10° C. SPW (0.52 Kg), trifluoroacetic acid (TFA) (29.38 Kg), and triisopropylsilane (TIS) (0.47 Kg) are charged into reactor II under nitrogen.

The mixed solution in reactor II is cooled to 0 to 10° C. and charged into reactor I at 25° C. The reaction mixture is stirred at 20-30 for 3 hr.

The reaction mixture is cooled to 0 to 10° C. and the pre-cooled (0 to 10° C.) methyl-t-butyl ether (MTBE) (61.85 Kg) is slowly charged at 15° C. and stirred for 1 hr. The solid product is filtered and washed with methyl-t-butylether (MTBE) twice and tetrahydrofuran (THF) twice. The wet cake is purged with nitrogen for 1 hr and dried at 50 for 6 hr to provide pramlintide acetate (about 1.21 Kg).

### Example 12

### Preparation of Protected Fragment S4 on resin (30-37, SPPS-Ligation Strategy)

### Fmoc-Thr(tBu)-Ans(Trt)-Val-Gly-Ser-(tBu)-Asn(Trt)-Thr(tBu)-Tyr(tBu)-Rink Amide MBHA Resin

Synthesis of the protected peptide was carried out by a stepwise Fmoc SPPS (solid phase peptide synthesis) procedure starting with loading FmocTyr(tBu)OH to the Rink Amide MBHA Resin (4-Methylbenzhydrylamine resin). After swelling and washing, the Fmoc protecting group of the Rink Amide MBHA Resin (60 g) was removed by treatment with 20% piperidine in DMF twice for 10 min and 30 min, respectively. The deprotected resin was washed for the excess deprotection reagent removal. The FmocTyr(tBu)OH (64.0 g) was activated in situ using 3:3:4.5 molar ratio of HBTU/HOBt/DIEA in DMF and subsequently coupled to the resin. The completion of the coupling reaction was indicated by Kaiser test, followed by excess reagents filtered off and the resin washed. The Fmoc protecting group on the α-amino group was removed with 20% piperidine in DMF twice for 10 min and 30 min, respectively. After washing of the resin, these steps were repeated each time with another amino acid according to peptide sequence. Trifunctional amino acids were side chain protected as follows: Tyr(tBu)-OH, Ser(tBu)-OH, Asn(Trt)-OH and Thr(tBu)-OH. Three equivalents of the activated amino acids were employed in the coupling reactions. At the end of the synthesis the growing peptide on resin was washed with DMF, MeOH followed by MTBE, and dried under vacuum to give dry peptide on resin: FmocThr(tBu)Ans(Trt)ValGlySer(tBu)Asn(Trt)Thr(tBu)Tyr(tBu)-Rink Amide MBHA Resin(S4-resin).

### Example 13

### De-Fmoc reaction (SPPS-Ligation Strategy)

42.4 g of Fmoc-Thr(tBu)-Asn(Trt)-Val-Gly-Ser(tBu)-Asn(Trt)-Thr(tBu)-Tyr(tBu)-Rink Amide MBHA Resin (S4-resin, 1.0 eq, base on blank resin 24 g) was swelled and washed by DMF. The Fmoc protecting group was removed by treatment with 20% piperidine in DMF twice for 10 min and 30 min, respectively. After washing of the resin, Thr(tBu)-Asn(Trt)-Val-Gly-Ser(tBu)-Asn(Trt)-Thr(tBu)-Tyr(tBu)-Rink Amide MBHA Resin (M9) was provided.

### Example 14

### Coupling Reaction (SPPS-Ligation Strategy)

S3: Fmoc-Ser(tBu)-Asn(Trt)-Phe-Gly-Pro-Ile-Leu-Pro-Pro-OH (28.2 g; 1.5 eq), 2-(1H-benzotriazole-1-yl)-1,1,3,3-tetramethylaminium hexafluorophosphate (HBTU) (5.87 g; 1.5 eq) and 1-hydroxy-7-azabenzotriazole (HOAt) (2.11 g; 1.5 eq) were charged into 1-methyl-2-pyrrolidinone (NMP) (240 ml) in a suitable reactor under nitrogen and stirred for 0.5 hr. The mixture was cooled to 0 to 10° C. and N—N′ diisopropylethylamine (DIEA) (4.1 mL; 2.25 eq) was charged into while maintaining the temperature at 0 to 10° C. and stirred for 5 min. The resulting solution was charged into a suitable reactor with M9. Completion of the coupling was indicated by a Kaiser test. After washing of the resin, FmocSer(tBu)Asn(Trt)PheGlyPro-IleLeuProProThr(tBu)Ans(Trt)ValGlySer(tBu)Asn(Trt)Thr(tBu)Tyr(tBu)-Rink Amide MBHA Resin (M10) was provided.

### Example 15

### De-Fmoc reaction (SPPS-Ligation Strategy)

The Fmoc group of M10 was removed by treatment with 20% piperidine in DMF twice for 10 min and 30 min, respectively. After washing of the resin, Ser(tBu)Asn(Trt)PheGlyProIleLeuProProThr(tBu)Ans(Trt)ValGlySer(tBu)Asn(Trt)Thr(tBu)Tyr(tBu)-Rink Amide MBHA Resin (M11) was provided.

### Example 16

### Coupling Reaction (SPPS Ligation Strategy)

S2: FmocThr(tBu)Gln(Trt)Arg(Pbf)LeuAlaAsn(Trt)PheLeuValHis(Trt)Ser(tBu)OH (53.7 g; 2.0 eq) and 1-hydroxy-7-azabenzotriazole (HOAt) (4.21 g; 3.0 eq) was charged into 1-methyl-2-pyrrolidinone (NMP) (264 ml) in a suitable reactor under nitrogen and stirred for 1 hr. The resulting solution was charged into M11 in a suitable reactor. The N,N-diisopropylcarbodiimide (DIC) (4.8 ml; 3.0 eq) was diluted with 1-methyl-2-pyrro-lidinone (NMP) (24 ml) and followed by dropped into reaction for 1 hr. The reaction mixture was stirred for 3 hr. Completion of the coupling was indicated by a Kaiser test. After washing of the resin, FmocThr(tBu)Glu(tBu)Arg(Pbf)LeuAlaAsn(Trt)PheLeuValHis(Trt)Ser(tBu)Ser(tBu)Asn(Trt)PheGlyProIleLeuProProThr(tBu)Ans(Trt)ValGlySer(tBu)Asn(Trt)Thr(tBu)Tyr(tBu)-Rink Amide MBHA Resin(M12) was provided.

### Example 17

### De-Fmoc reaction(SPPS Ligation Strategy)

The Fmoc group of M12 was removed by treatment with 20% piperidine in DMF twice for 10 min and 30 min, respectively. After washing of the resin, Thr(tBu)Glu(tBu)Arg(Pbf)LeuAlaAsn(Trt)PheLeuValHis(Trt)Ser(tBu)Ser(tBu)Asn(Trt)PheGlyProIleLeuProProThr(tBu)Ans(Trt)ValGlySer(tBu)Asn(Trt)Thr(tBu)Tyr(tBu)-Rink Amide MBHA Resin(M13) was provided.

### Example 18

### Coupling Reaction (SPPS Ligation Strategy)

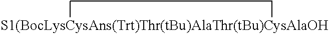

(21.1 g; 1.5 eq) and 1-hydroxy-7-azabenzotriazole (HOAt) (3.16 g; 2.25 eq) was charged into 1-methyl-2-pyrrolidinone (NMP) (240 ml) in a suitable reactor under nitrogen and stirred for 0.5 hr. The resulting solution was charged into M13 in a suitable reactor. The N,N-diisopropylcarbodiimide (DIC) (3.6 ml; 2.25 eq) was diluted with 1-methyl-2-pyrrolidinone (NMP) (24 ml) and followed by dropped into reaction for 1 hr. The reaction mixture was stirred for 3 hr. The Completion of the coupling was indicated by a Kaiser test. After washing of the resin,

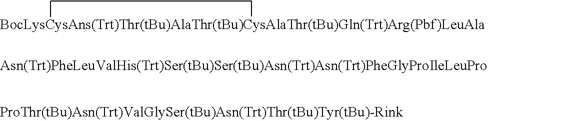

Amide MBHA Resin (M14) was provided.

### Example 19

### Deblocking Reaction (SPPS Ligation Strategy)

Trifluoroacetic acid (TFA) (604.8 ml), triisopropylsilane (TIS) (13.4 ml), and ethanethiol (EtSH) (47 ml) was charged into SPW (6.72 ml) in a suitable reactor under nitrogen. The mixed solution was pre-cooled to 0 to 10° C. followed by charged into M14 (84 g) in a suitable reactor under nitrogen. The reaction mixture was allowed to return to 20-30° C. and stirred for 2 hr. The reaction solution was drained for collection. The collected reaction solution was further cooled to 0 to 10° C. and pre-cooled (0 to 10° C.) methyl-t-butyl ether (MTBE) (4.2 L) was slowly charged at 15° C. and stirred for 1 hr. The solid product was collected by filtration and washed with methyl-t-butylether (MTBE) twice and tetrahydrofuran (THF) twice. The wet cake is purged with nitrogen for 1 hr and dried at 50° C. for 6 hr to provide pramlintide (about 38 g).

We claim:

1. A process for the preparation of pramlintide of formula (I): 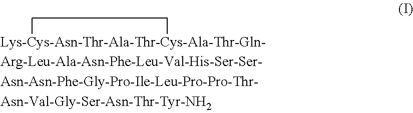
comprising:
(a) preparing a protected side chain peptide of formula (II):
P1-Thr(P2)-Asn(P2)-Val-Gly-Ser(P2)-Asn(P2)-Thr(P2)-Tyr(P2)-resin (II),
wherein P1 and P2 are protecting groups;
(b) removing the terminal P1 protecting group and reacting with a protecting side chain peptide of formula (III)
P1-Ser(P2)-Asn(P2)-Asn(P2)-Phe-Gly-Pro-Ile-Leu-Pro-Pro-OH (III)
wherein P1 and P2 are as defined above, to yield a protected side chain peptide of formula (IV)
P1-Ser(P2)-Asn(P2)-Asn(P2)-Phe-Gly-Pro-Ile-Leu-Pro-Pro-Thr(P2)-Asn(P2)-Val-Gly-Ser(P2)-Asn(P2)-Thr(P2)-Tyr(P2)-resin; (IV);
(c) removing the terminal P1 protecting group and reacting with a protecting side chain peptide of formula (V)
P1-Thr(P2)-Gln(P2)-Arg(P2)-Leu-Ala-Asn(P2)-Phe-Leu-Val-His(P2)-Ser(P2)-OH (V)
wherein P1 and P2 are as defined above, to yield a protected side chain peptide of formula (VI)
P1-Thr(P2)-Gln(P2)-Arg(P2)-Leu-Ala-Asn(P2)-Phe-Leu-Val-His(P2)-Ser(P2)-Ser(P2)-Asn(P2)-Asn(P2)-Phe-Gly-Pro-Ile-Leu-Pro-Pro-Thr(P2)-Asn(P2)-Val-Gly-Ser(P2)-Asn(P2)-Thr(P2)-Tyr(P2)-resin; (VI);
(d) removing the terminal P1 protecting group and reacting a protecting side chain peptide of formula (VII) 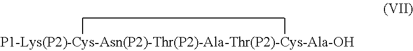
wherein P1 and P2 are as defined above, to yield a protected side chain pramlintide of formula (VIII) 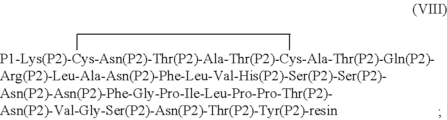
(e) removing the resin and each of the protecting groups P1 and P2 to yield the pramlintide of formula (I).

2. The process of claim 1, wherein P1 is Fmoc or Boc.

3. The process of claim 1, wherein P1 of formula (VII) in step (d) is Boc.

4. The process of claim 1, wherein P2 is selected from tBu, Trt and Pbf

5. The process of claim 1, wherein steps (a) to (e) are performed on a solid support.

6. The process of claim 5, wherein the solid support is 4-methylbenzhydrylamine resin.

7. The process of claim 1, wherein at least one of the coupling reaction of steps a) to d) is accomplished in the presence of a reagent selected from 1-hydroxy-7-azabenzotriazole, 1-methyl-2-pyrrolidinone, 2-(1H-benzotriazole-1-yl)-1,1,3,3-tetramethylaminium hexafluorophosphate, diisopropylethylamine, N,N-diisopropylcarbodiimide, and combinations thereof.

8. The process of claim 1, wherein the deprotecting step is carried out in the presence of piperidine when the protecting group is Fmoc, or in the presence of trifluoroacetic acid when the protecting group is Boc.

9. A protected side chain peptide of formula (II)
P1-Thr(P2)-Asn(P2)-Val-Gly-Ser(P2)-Asn(P2)-Thr(P2)-Tyr(P2)-resin; (II)
wherein P1 and P2 are protecting groups.

10. The peptide of claim 9, which is Fmoc-Thr(tBu)Asn(Trt)ValGlySer(tBu)Asn(Trt)Thr(tBu)Tyr(tBu)-resin.

11. The peptide of claim 10, wherein
Fomc-Thr(tBu)Asn(Trt)ValGlySer(tBu)Asn(Trt)Thr(tBu)Tyr(tBu)-resin is produced by solid phase synthesis comprising coupling a protected designated amino acid to a growing peptide chain covalently linked to an insoluble solid resin support to give Fomc-Thr(tBu)Asn(Trt)ValGlySer(tBu)Asn(Trt)Thr(tBu)Tyr(tBu)-resin.

12. A protected side chain peptide of formula (III)
P1-Ser(P2)-Asn(P2)-Asn(P2)-Phe-Gly-Pro-Ile-Leu-Pro-Pro-OH (III),
wherein P1 and P2 are protecting groups.

13. The peptide of claim 12, which is Fmoc-Ser(tBu)-Asn(Trt)-Asn(Trt)-Phe-Gly-Pro-Ile-Leu-Pro-Pro-OH.

14. The peptide of claim 13 wherein the peptide is made by a process comprising coupling a protected designated amino acid to a growing peptide chain covalently linked to an insoluble solid resin support to give Fmoc-Ser(tBu)-Asn(Trt)-Asn(Trt)-Phe-Gly-Pro-Ile-Leu-Pro-Pro-OH.

15. A protected side chain peptide of formula (V)
P1-Thr(P2)-Gln(P2)-Arg(P2)-Leu-Ala-Asn(P2)-Phe-Leu-Val-His(P2)-Ser(P2)-OH (V),
wherein P1 and P2 are protecting groups.

16. The peptide of claim 15, which is Fmoc-Thr(tBu)-Gln(Trt)Arg(Pbf)-Leu-Ala-Asn(Trt)-Phe-Leu-Val-His(Trt)-Ser(tBu)-OH.

17. The peptide of claim 16 wherein the peptide is made by a process comprising coupling a protected designated amino acid to a growing peptide chain covalently linked to an insoluble solid resin support to give Fmoc-Thr(tBu)-Gln(Trt)Arg(Pbf)-Leu-Ala-Asn(Trt)-Phe-Leu-Val-His(Trt)-Ser(tBu)-OH and a deprotecting step.

18. A protected side chain peptide of formula (VII) 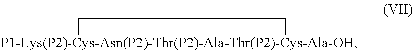
wherein P1 and P2 are protecting groups.

19. The peptide of claim 18, which is

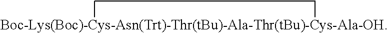

20. The peptide of claim 19 wherein the peptide is made by a process comprising coupling a protected designated amino acid to a growing peptide chain covalently linked to an insoluble solid resin support and selectively deprotecting the cysteine residues and forming an intramolecular disulfide bond between cysteine residues on the peptide chain before cleaving the peptide chain from the solid support to produce

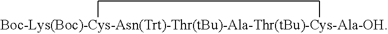

21. A process for the preparation of pramlintide of formula (I): 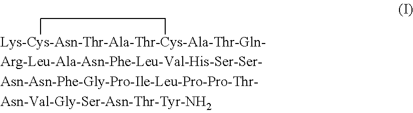
comprising:
(a) reacting a protected side chain peptide of formula (IIA):
P1-Thr(P2)-Asn(P2)-Val-Gly-Ser(P2)-Asn(P2)-Thr(P2)-OH (IIA)
wherein P1 and P2 are protecting groups, with H-Tyr(P2)-NH₂ to yield a protected side chain peptide of formula (IIIA)
P1-Thr(P2)-Asn(P2)-Val-Gly-Ser(P2)-Asn(P2)-Thr(P2)-Tyr(P2)-NH₂ (IIIA);
(b) removing the terminal P1 protecting group and reacting with a protecting side chain peptide of formula (IVA)
P1-Ser(P2)-Asn(P2)-Asn(P2)-Phe-Gly-Pro-Ile-Leu-Pro-Pro-OH (IVA)
wherein P1 and P2 are as defined above, to yield a protected side chain peptide of formula (VA)
P-Ser(P2)-Asn(P2)-Asn(P2)-Phe-Gly-Pro-Ile-Leu-Pro-Pro-Thr(P2)-Asn(P2)-Val-Gly-Ser(P2)-Asn(P2)-Thr(P2)-Tyr(P2)-NH₂ (VA);
(c) removing the terminal P protecting group and reacting with a protecting side chain peptide of formula (VIA)
P1-Thr(P2)-Gln(P2)-Arg(P2)-Leu-Ala-Asn(P2)-Phe-Leu-Val-His(P2)-Ser(P2)-OH (VIA)
wherein P1 and P2 are as defined above, to yield a protected side chain peptide of formula (VIIA)
P1-Thr(P2)-Gln(P2)-Arg(P2)-Leu-Ala-Asn(P2)-Phe-Leu-Val-His(P2)-Ser(P2)-Ser(P2)-Asn(P2)-Asn(P2)-Phe-Gly-Pro-Ile-Leu-Pro-Pro-Thr(P2)-Asn(P2)-Val-Gly-Ser(P2)-Asn(P2)-Thr(P2)-Tyr(P2)-NH₂ (VIIA);
(d) removing the terminal P1 protecting group and reacting a protecting side chain peptide of formula (VIIIA) 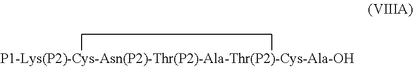
wherein P1 and P2 are as defined above, to yield a protected side chain pramlinitide of formula (IXA) 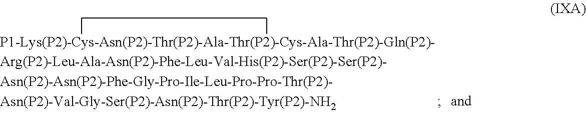
(e) removing each of the protecting groups P1 and P2 to yield the pramlinitide of formula (I).

22. The process of claim 21, wherein steps (a) to (e) are performed in solution.
